(19) United States
(12) Patent Application Publication
(10) Pub. No.: US 2015/0297150 A1
(43) Pub. Date: Oct. 22, 2015

(54) Mammography Apparatus and Method to Monitor and/or Adjust the Settings of Such a Mammography Apparatus

(71) Applicant: Academisch Medisch Centrum bij de Universiteit van Amsterdam, Amsterdam (NL)

(72) Inventors: Cornelis Antonius Grimbergen, Amsterdam (NL); Gerardus Johannes Den Heeten, Amsterdam (NL)

(21) Appl. No.: 14/788,208

(22) Filed: Jun. 30, 2015

Related U.S. Application Data
(63) Continuation of application No. PCT/NL2014/050001, filed on Jan. 6, 2014.

(30) Foreign Application Priority Data
Jan. 8, 2013 (NL) 2010095

(51) Int. Cl. A61B 6/04; A61B 6/00

(52) U.S. Cl. CPC A61B 6/0414; A61B 6/502; A61B 6/0435; A61B 6/44; A61B 6/586

(57) ABSTRACT

Method and apparatus for monitoring and/or adjusting the settings of a mammography apparatus comprising a mainframe, a subframe mounted in the mainframe, wherein the subframe supports an x-ray source, a paddle and a detector with a detector cover, including providing of a suspension for connecting the paddle with the subframe, wherein the suspension of the paddle is provided with force sensors for obtaining a measurement signal that is indicative of an amount of compression applied with the paddle to a breast.

### CROSS-REFERENCE TO RELATED APPLICATIONS

This application is a continuation application of Patent Cooperation Treaty Application No. PCT/NL2014/050001, filed on Jan. 6, 2014, which claims priority to Netherlands Patent Application No. 2010095, filed on Jan. 8, 2013, and the specifications and claims thereof are incorporated herein by reference.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### COPYRIGHTED MATERIAL

Not Applicable.

### BACKGROUND OF THE INVENTION

1. Field of the Invention (Technical Field)

The present invention relates to a mammography apparatus comprising a mainframe and a subframe mounted in the mainframe, which subframe supports an x-ray source, a paddle and a detector with a detector cover, and which paddle is connected with the subframe through a suspension.

The invention further relates to a method to monitor and/or adjust the settings of such a mammography apparatus.

2. Description of Related Art

A mammography apparatus is known from WO2011/102713.

In such a known mammography apparatus it is day to day practice to place and compress a patient's breast between the detector and the paddle before executing an x-ray measurement in order to optimize the image quality and minimize the required x-ray doses. During the procedure the patient is standing next to the mammograph. The compression is applied by bringing the patient's breast in between the x-ray detector and the paddle, which paddle can thereafter be lowered in the direction and relative to the detector by hand or by activating a motor built in the mammography apparatus. Generally speaking there is a minimum pressure required to provide sufficient flattening to the breast to suit the requirements of the x-ray imaging. For safety reasons there is a maximum amount of force that can be applied which is set at a value of 20 daN. Consequently a wide variety of pressures can be applied depending on the contact area of the compressed breast on which the force is exercised. For that reason the upper limit of compression that can be applied depends on the size of the women's breasts. It is commonly known that this procedure may inflict serious pain on the patient. Also bruises and hematomas are reported on a regular base. To alleviate these problems it is important that the amount of pressure applied to the breast remains restricted. There are, however, situations during examination that may require that the pressure applied to the breast is increased in order to meet the required x-ray imaging conditions. This may for instance occur during a cranio caudal examination of the breast when a skin crease or stretch of the skin reduces the effectivity of the applied pressure. It may also occur during a medial lateral oblique examination of the breast when the paddle pressure is applied in part to the shoulder area and/or the breast muscle. Also this adversely affects the effective pressure applied to the breast. In the prior art these problems are addressed by increasing the pressure applied to the breast, therewith worsening the pain sensations and inflicting other adverse effects for the patient.

### BRIEF SUMMARY OF THE INVENTION

It is one of the objectives of the invention to improve the effectivity of the pressure applied to the breast.

It is a further objective of the invention to reduce the patient's experience of pain.

It is another objective of the invention to improve the reliability and repeatability of the x-ray measurement by providing a more reliable and reproducible compression of the breast.

It is still another objective of the invention to provide an improved mammography apparatus which ensures that the objectives of an optimal trade-off between reduced pain and more reliable and repeatable x-ray measurements can be achieved.

Further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more preferred embodiments of the invention and are not to be construed as limiting the invention. In the drawings:

FIG. 1 shows schematically a cranio caudal breast examination;

FIG. 2 shows schematically a medial lateral oblique examination of the breast;

FIGS. 3A and 3B show schematically a frontal view and a side view respectively, of a mammography apparatus ac-cording to the invention;

FIG. 4 shows a first embodiment of the mammography apparatus of the invention wherein the force sensors are applied at the back rods of the suspension; and

FIG. 5 shows a second embodiment of the mammography apparatus of the invention wherein the force sensors are applied at the side rods of the suspension.

### DETAILED DESCRIPTION OF THE INVENTION

According to a first aspect of the invention, the suspension of the paddle comprises force sensors for providing a measurement signal that is indicative of an amount of compression applied with the paddle to a breast. Providing the force sensors in the suspension of the paddle ensures that a very accurate and reliable measurement of the compression applied to the breast can be obtained.

Preferably there is at least a first force sensor and a second force sensor, that are placed at mirror image positions with reference to the subframe. This makes the measurement possible of the forces that are applied to the left and right side of the patient's breast.

Preferably further a measurement and control unit is provided to actuate a drive motor for controlling an inclination of the paddle with reference to the detector cover depending on the measurement signals from the force sensors. In this way a desirable distribution of forces along the breast can be obtained.

In one embodiment the measurement and control unit is arranged to actuate the drive motor for controlling an inclination of the paddle with reference to the detector cover so as to equalize the forces as measured with the respective force sensors. This provides the best estimate for a uniform distribution of forces along the breast during compression and imaging, can provide important diagnostic information and can be helpful in mitigating the pain that the patient experiences to the least possible extent.

The force sensors are advantageously placed at a position to prevent interference with the X-ray beam. This makes possible that a force exerted with the paddle during pressurizing of a breast is measured with said force measurement means during pressurizing and imaging so as to enable balancing or equalizing the force exerted on the contact area between the paddle and the breast during the imaging process.

The balancing of the measured forces can be done manually or automatically, in the latter case for instance using springs or a motor drive.

Particularly when manual adjustment of the mammography apparatus is carried out, it is advantageous that the measurement and control unit is arranged to indicate a differential force of the first force sensor and the second force sensor that are placed on mirror image positions with respect to the subframe. This may of course also be applied when the balancing of the measured forces is carried out automatically.

As mentioned the measurement and control unit is arranged to actuate a drive motor for controlling an inclination of the paddle with reference to the detector cover depending on the measurements with the force sensors provided at mirror image positions with respect to the subframe so as to automatically equalize the forces as measured with these respective force sensors. This adjustment of the inclination of the paddle with reference to the detector cover will be limited to only a few degrees to maintain image quality, but enough to largely circumvent the influence of the earlier mentioned skin crease or the shoulder or breast muscle. This may apply both to a cranio caudal examination and with a medial lateral oblique examination of the breast.

Furthermore the distribution of forces as measured with the force sensors during compression may indicate important differences in tissue stiffness, which can indicate a mass which can potentially be invisible in the mammographic image. If the differential force between the forces measured by the first force sensor and by the second force sensor exceeds a predefined value, preferably an indicator signal is provided to warn an operator of the mammography apparatus that repositioning of the breast is indicated. If the disbalance persists the information can be transferred to the radiologist. The disbalance can for instance be expressed as the ratio between the measured forces at or near the respective sides of the paddle. The warning signal is particularly useful to indicate the operator that on one side of the paddle the compression is less or not useful and the desired goal of the compression i.e. flattening and immobilization of the breast during exposure cannot be achieved, and repositioning of the breast should be considered.

Whenever in the figures the same reference numerals are applied, these numerals refer to the same parts.

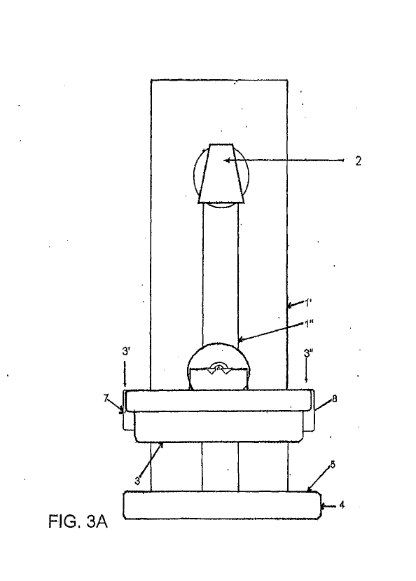

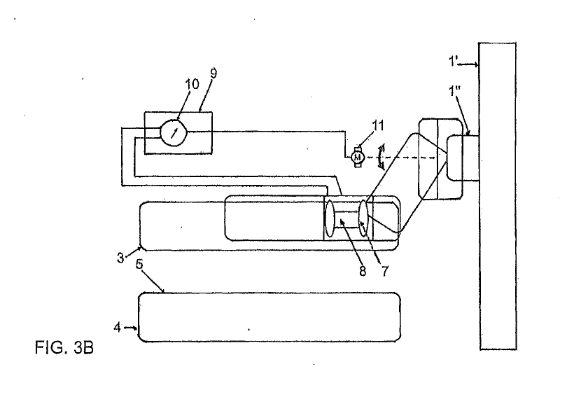

With reference first to FIG. 3A a frontal view is shown of a mammography apparatus 1 which has a mainframe 1′ that is provided with an x-ray source 2, a paddle 3 and a detector 4 with a detector cover 5 that during use contacts the underside of a patient's breast. The person skilled in the art is aware that the x-ray source 2, the paddle 3 and the detector 4 are placed on a subframe 1″ (also called c-arm) that is mounted in the mainframe 1′. The subframe 1″ may be moved up and down and is rotatable.

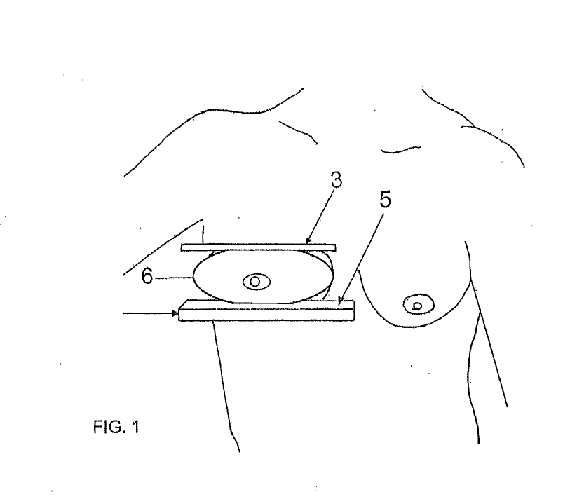

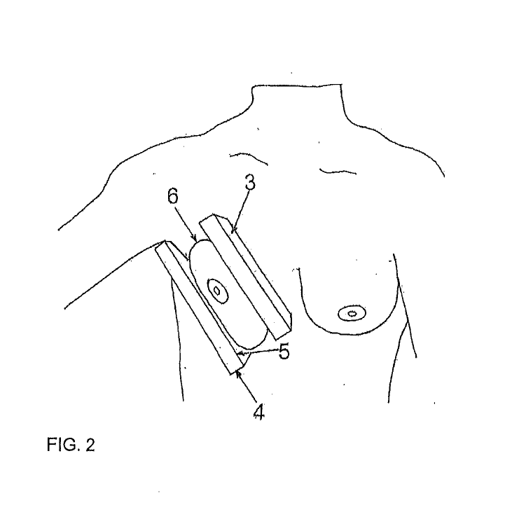

With reference to FIG. 1 a cranio caudal examination of a patient's breast 6 is shown, wherein for clarity only the paddle 3, the detector 4 and the detector cover 5 are shown with reference to said breast 6. In FIG. 2 the so-called medial lateral oblique examination of the breast 6 is shown, which is featured by the oblique positioning of the subframe 1″ with reference to the frame 1′ to cause that the paddle 3 and the detector 4 with the detector cover 5 are placed at an angle with reference to the horizontal.

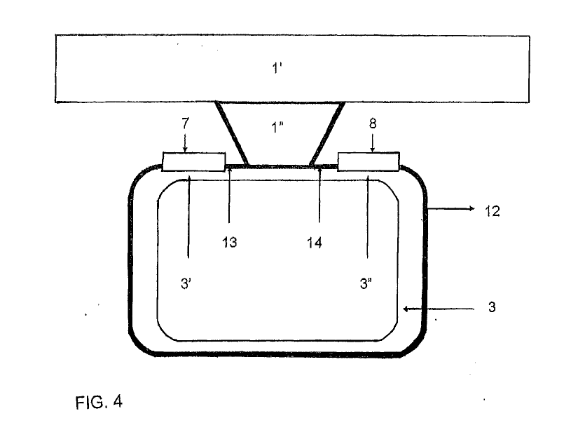

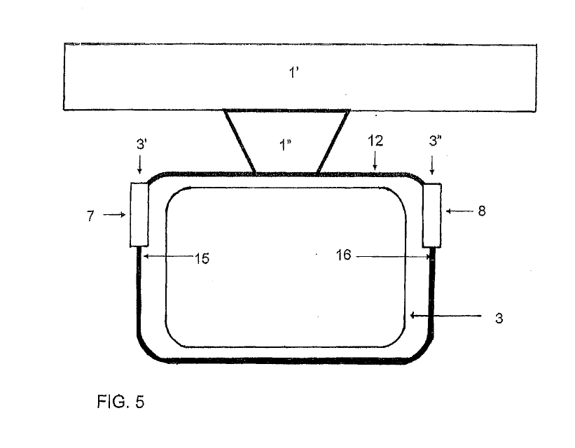

Turning again to FIG. 3A and also making reference to FIGS. 4 and 5 it is shown that the paddle 3 has measurement means embodied as force sensors 7, 8 that are provided at positions 3′, 3″ as part of the suspension 12 of the paddle 3 and because thereof outside the X-ray beam originating from x-ray source 2. In FIG. 4 two force sensors 7, 8 are shown positioned in the back rods 13, 14 of the suspension 12; in FIG. 5 two force sensors 7, 8 are shown positioned in the side rods 15, 16 of the suspension 12. Both embodiments of FIG. 4 and FIG. 5 enable that the force that is exerted with the paddle 3 during pressurizing of a breast 6 is measured with said force sensors 7, 8 so that these respective forces at both sides of the paddle 3 can be balanced or equalized.

FIG. 3B further shows that the mammography apparatus 1 according to the shown preferred embodiment is further provided with a measurement and control unit 9 that is connected to the first force sensor 7 which is placed at a first position 3′ in the suspension 12 of the paddle 3 and to the second force sensor 8 which is placed at a second position 3′ in the suspension 12 of the paddle 3. The two said positions 3′, 3″ are mirror image positions with reference to the subframe 1″. The measurement and control unit 9 is further arranged to indicate, preferably with indicator means 10, a differential force of the first force sensor 7 and the second force sensor 8. This can then be suitably used to actuate the paddle either manually or automatically depending on the measurements with the force sensors 7, 8 provided at the mirror image positions 3′, 3″ in the suspension 12 of the paddle 3.

FIG. 3B shows that the measurement and control unit 9 is arranged to actuate a drive motor 11 for controlling an inclination of the paddle 3 with reference to the detector cover 5 depending on the measurements with the force sensors 7, 8 provided at the mirror image positions 3′, 3″ in the suspension 12 of the paddle 3 so as to equalize the forces as measured with these respective force measurement means 7, 8.

It is emphasized that the invention should not be deemed restricted to the exemplary embodiment of a mammography apparatus according to the invention with reference to the drawing. This exemplary embodiment merely serves to elucidate the wording as used in the appended claims. The protective scope that merits the invention is, however, exclusively determined by the appended claims, wherein the meaning of the wording used in the claims can be explained with reference to the description and this exemplary embodiment.

Although the invention has been described in detail with particular reference to these preferred embodiments, other embodiments can achieve the same results. Variations and modifications of the present invention will be obvious to those skilled in the art and it is intended to cover in the appended claims all such modifications and equivalents. The entire disclosures of all references, applications, patents, and publications cited above are hereby incorporated by reference.

What is claimed is:

1. A mammography apparatus comprising a mainframe and a subframe mounted in the mainframe, which subframe supports an x-ray source, a paddle and a detector with a detector cover, and which paddle is connected with the subframe through a suspension, wherein the suspension of the paddle comprises force sensors for providing a measurement signal that is indicative of an amount of compression applied with the paddle to a breast.

2. The mammography apparatus according to claim 1, wherein there is at least a first force sensor and a second force sensor, that are placed at mirror image positions with reference to the subframe.

3. The mammography apparatus according to claim 1, wherein a measurement and control unit is provided to actuate a drive motor for controlling an inclination of the paddle with reference to the detector cover depending on the measurement signals from the force sensors.

4. The mammography apparatus according to claim 3, wherein the measurement and control unit is arranged to actuate the drive motor for controlling an inclination of the paddle with reference to the detector cover so as to equalize the forces as measured with the respective force sensors.

5. The mammography apparatus according to claim 1, wherein the force sensors are placed at a position within the suspension of the paddle to prevent interference with an x-ray beam from the x-ray source.

6. The mammography apparatus according to claim 3, wherein the measurement and control unit is arranged to indicate a differential force of the first force sensor and the second force sensor.

7. A method for monitoring and/or adjusting the settings of a mammography apparatus comprising a mainframe, a subframe mounted in the mainframe, wherein the subframe supports an x-ray source, a paddle and a detector with a detector cover, comprising providing a suspension for connecting the paddle with the subframe and providing the suspension of the paddle with force sensors for obtaining a measurement signal that is indicative of an amount of compression applied with the paddle to a breast.

8. The method according to claim 7, comprising providing the force sensors within the suspension of the paddle on mirror image positions with reference to the subframe.

9. The method according to claim 7, comprising providing a drive motor for controlling an inclination of the paddle with reference to the detector cover depending on the measurements with the force sensors.

10. The method according to claim 9, comprising providing that the drive motor is actuated for controlling an inclination of the paddle with reference to the detector cover depending on the measurements with the force sensors so as to equalize the forces as measured with these respective force sensors.

11. The method according to claim 7, comprising providing the force sensors at a position within the suspension of the paddle to prevent interference with an x-ray beam from the x-ray source.

12. The method according to claim 7, comprising providing that the forces measured with the respective force sensors are compared to indicate a differential force between said forces measured by the force sensors.

13. The method according to claim 7, comprising providing that the paddle is actuated manually or automatically depending on the measurements with the force sensors.

14. The method according to claim 7, comprising providing an indicator signal to warn an operator of the mammography apparatus that repositioning of the breast is indicated if a differential force between the forces measured by the force sensors that are placed on mirror image positions with respect to the subframe, exceeds a predefined value.
